(19) United States
(12) Patent Application Publication
(10) Pub. No.: US 2024/0050442 A1
(43) Pub. Date: Feb. 15, 2024

(54) TRIMEPRAZINE DERIVATIVES AND COMPOSITIONS COMPRISING THE SAME FOR TREATING A NEUROPSYCHIATRIC DISORDER

(71) Applicants: CENTRE NATIONAL DE LA RECHERCHE SCIENTIFIQUE, PARIS (FR); UNIVERSITÉ COTE D'AZUR, NICE (FR)

(72) Inventors: BARBARA BARDONI, NICE (FR); THOMAS MAURIN, ANTIBES (FR)

(21) Appl. No.: 18/265,270

(22) PCT Filed: Dec. 7, 2021
(86) PCT No.: PCT/EP2021/084484

§ 371 Date: Jun. 5, 2023

(30) Foreign Application Priority Data
Dec. 7, 2020 (EP) 20306508.1

(51) Int. Cl. A61K 31/5415; A61K 9/00; A61P 25/00

(52) U.S. Cl. CPC A61K 31/5415; A61K 9/0019; A61P 25/00

(57) ABSTRACT

The present invention relates to trimeprazine derivatives of formula (I) for use for treating a neuropsychiatric disorder chosen among an intellectual disability (ID), preferably fragile X syndrome (FXS), autism spectrum disorders (ASD), and attention deficit hyperactivity disorder (ADID). The invention also relates to pharmaceutical compositions for use comprising such derivatives.

### TECHNICAL FIELD

The present invention relates to the medical field, and more particularly, to the field of diseases and disorders of the nervous system such as an intellectual disability, preferably fragile X syndrome (FXS), autism spectrum disorders (ASD), and attention deficit hyperactivity disorder (ADHD).

### TECHNICAL BACKGROUND

Diseases and disorders of the nervous system create a significant burden of morbidity and mortality worldwide. Fragile X syndrome (FXS) is a rare genetic disease that affects 10000 patients in France and more than 150000 in Europe. FXS is the most common form of hereditary intellectual disability and one of the main monogenic causes of autism. FXS results from the absence of expression of the gene FMR1 and its encoded protein FMRP. At this date, FXS remains an unmet medical need because there is no available specific therapy for this disorder that further share the same symptoms with autism. Current therapeutic approaches rather focus on behavioural therapies including psychological intervention and drugs therapy, which are mainly used to treat limited symptoms such as hyperactivity, stress, and anxiety.

Therefore, novel therapeutic strategies for treating FXS, autism, and more generally neuropsychiatric disorders, must be developed. In this context, Wiley et al. (Journal of Pharmaceutics & Drug Delivery Research, 2015, 4:3) have targeted the histamine route and have disclosed the use of hydroxyzine, a Histamine Receptor 1 antagonist, for treating Autism Spectrum Disorders. However, H1R target needs to be more investigated to provide improved treatments against the neuropsychiatric disorders.

It thus remains a need for developing news drugs exhibiting an improved therapeutic effect against neuropsychiatric disorders, at particular intellectual disability (ID), fragile X syndrome (FXS), autism spectrum disorders (ASD), and attention deficit hyperactivity disorder (ADHD), preferably while avoiding side effects for the patient, such as for instance falling asleep. The present invention seeks to meet these and other needs.

### SUMMARY OF THE INVENTION

The inventors have evaluated two H1R antagonists, namely trimeprazine and opipramol, using referenced tests on social behaviours and cognition, which model an intellect disability, such as FXS, autism spectrum disorders (ASD), and attention deficit hyperactivity disorder (ADHD). Unexpectedly, the inventors have shown an improved effect for trimeprazine whereas the results with opipramol were disappointed with no beneficial or negative impact.

Accordingly, the invention concerns trimeprazine derivatives for use for treating a neuropsychiatric disorder such as an intellectual disability (ID), preferably fragile X syndrome (FXS), autism spectrum disorders (ASD), and attention deficit hyperactivity disorder (ADHD).

The present invention thus relates to a compound of formula (I):

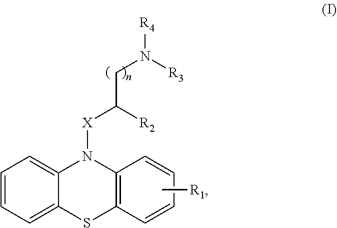

wherein:
X is —CO— or —CH₂—
n is 0 or 1;
R₁ represents:
   a hydrogen,
   a —CO—R₅ with R₅ being a hydrogen or a (C₁-C₆)alkyl,
   a (C₁-C₆)alkyl optionally substituted by at least one halogen,
   a (C₁-C₆)alkyloxy optionally substituted by at least one halogen,
   a halogen, or
   a —SO₂—NR₆R_{6′} with R₆ and R_{6′} being independently a hydrogen or a (C₁-C₆)alkyl;
R₂ represents a hydrogen or a (C₁-C₆)alkyl; and
R₃ and R₄ represent independently a hydrogen or a (C₁-C₆)alkyl;

or one of its stereoisomers, and the pharmaceutically acceptable salts thereof, for use for treating a neuropsychiatric disorder chosen among an intellectual disability (ID), preferably fragile X syndrome (FXS), autism spectrum disorders (ASD), attention deficit hyperactivity disorder (ADHD). Preferably, said neuropsychiatric disorder is fragile X syndrome (FXS) and autism spectrum disorders (ASD).

In a particular embodiment, a compound of formula (I) for use according to the invention is such that:
X is —CO— or —CH₂—
n is 0 or 1;
R₁ represents:
   a hydrogen,
   a —CO—R₅ with R₅ being (C₁-C₃)alkyl, preferably a methyl or an ethyl,
   a methyl substituted by at least one fluorine, preferably a —CF₃,
   a methoxy,
   a chlorine, or
   a —SO₂—NR₆R_{6′} with R₆ and R_{6′} being a methyl;
R₂ represents a hydrogen or a methyl; and
R₃ and R₄ represent independently a (C₁-C₆)alkyl, preferably a methyl or an ethyl.

In a particular aspect, X is —CH₂—. In a further particular aspect, n is 1. In a further particular aspect, R₁ is H. In a further particular aspect, R₂ is a methyl. In a further particular aspect, R₃ and R₄ represent a methyl.

A preferred compound of formula (I) for use according to the invention is a compound selected in a group consisting of trimeprazine, acepromazine, aceprometazine, chlorpromazine, dacemazine, dimetotiazine, fluacizine, levomeprazine, profenamine, promazine, promethazine, propiomazine, triflupromazine, and a pharmaceutical acceptable salt thereof. More preferably, said compound of formula (I) for use is trimeprazine or one of its pharmaceutical acceptable salt, even more preferably trimeprazine tartrate.

Another object of the invention is a pharmaceutical composition comprising a compound as defined herein and a pharmaceutically acceptable excipient, for use for treating a neuropsychiatric disorder chosen among an intellectual disability (ID), preferably fragile X syndrome (FXS), autism spectrum disorders (ASD), attention deficit hyperactivity disorder (ADHD). In a preferred embodiment, said pharmaceutical composition for use comprises trimeprazine or one of its pharmaceutical acceptable salt as a sole active ingredient.

In a particular embodiment, the pharmaceutical composition for use according to the invention is administered in a mammal at a dose of trimeprazine or one of its pharmaceutical acceptable salt ranging from 0.01 to 0.50 mg/kg BW, from 0.10 to 0.40 mg/kg BW, preferably from 0.20 to 0.30 mg/kg BW, even more preferably at a dose of 0.25 mg/kg BW. In a further particular embodiment, the pharmaceutical composition for use according to the invention is administered by oral or parenteral route, preferably by intraperitoneal route. Preferably, the mammal is a human, preferably an infant, an adolescent, or an adult.

### LEGEND OF FIGURES

FIG. 1: Impact of trimeprazine (SM4) on the social behaviour of infant and adolescent Fmr1-KO mice. FIG. 1A: Homing Behaviour test—Social discrimination, Cognition—Infant mice 13 post-natal days. SM4: 0.25 mg/kg BW. Latency time is indicated in second. Error bars indicate SEM. Two-Way Anova was applied and post-hoc Tukey test for multiple comparisons (n=6-10 for each group). *p≤0.05. Interaction F(1,27)=7.162 P=0.0125. FIG. 1B: Social interaction test—Adolescent mice 28-30 post-natal days. SM4: 0.25 mg/kg BW. Number of social interactions is indicated. Error bars indicate SEM. Two-Way Anova was applied and post-hoc Tukey test for multiple comparisons (n=6-11 for each group). *** p≤.,001; *p≤0.05. Interaction F (1,31)=4.649 P=0.0389.

FIG. 2: Impact of opipramol (SM2) on the social behaviour of infant and adolescent Fmr1-KO mice. FIG. 2A: Homing behaviour test—Social discrimination, Cognition—Infant mice 13 post-natal days. SM2: 0.175 mg/kg BW. Latency time is indicated in second. Error bars indicate SEM. Two-Way Anova was applied and post-hoc Tukey test for multiple comparisons (n=4-8 for each group). **p≤0.01. FIG. 2B: Social interaction test —Adolescent mice 28-30 post-natal days. SM2: 0.25 mg/kg BW. Number of social interactions is indicated. Error bars indicate SEM. Two-Way Anova was applied and post-hoc Tukey test for multiple comparisons (n=3-12 for each group). ** p≤0.01; *p≤0.05.

FIG. 3: Impact of trimeprazine (SM4) and opipramol (SM2) on the behaviour of adult Fmr1-KO mice. FIG. 3A: Recognition (Cognition), Adult mice 8 weeks, SM4: 0.3 mg/kg BW. Error bars indicate SEM. Two-Way Anova was applied and post-hoc Tukey test for multiple comparisons (n=5-7 for each group). **p≤0.01; Interaction: F(1,22)=4.382 p=0.0481. FIG. 3B: Recognition (Cognition), Adult mice 8 weeks, SM2: 0.18 mg/kg BW. Error bars indicate SEM. Two-Way Anova was applied and post-hoc Tukey test for multiple comparisons (n=5-10 for each group). *p≤0.05; ***p≤0,001; ****p≤0,0001; Interaction: F(1,26)=5,473 p=0.0273.

### DETAILED DESCRIPTION OF THE INVENTION

### Definitions

According to the present invention, the terms below have the following meanings: The terms mentioned herein with prefixes such as for example C₁-C₃, C₁-C₆ or C₂-C₆ can also be used with lower numbers of carbon atoms such as C₁-C₂, C₁-C₅, or C₂-C₅. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms.

The term “alkyl” refers to a saturated, linear or branched aliphatic group. The term “(C₁-C₃)alkyl” more specifically means methyl, ethyl, propyl, or isopropyl. The term “(C₁-C₆)alkyl” more specifically means methyl, ethyl, propyl, isopropyl, butyl, isobutyl, tert-butyl, pentyl or hexyl. In a preferred embodiment, the “alkyl” is a methyl, an ethyl, a propyl, an isopropyl, or a tert-butyl, more preferably a methyl.

The term “alkoxy” or “alkyloxy” corresponds to the alkyl group as above defined bonded to the molecule by an —O— (ether) bond. (C₁-C₃)alkoxy includes methoxy, ethoxy, propyloxy, and isopropyloxy. (C₁-C₆)alkoxy includes methoxy, ethoxy, propyloxy, isopropyloxy, butyloxy, isobutyloxy, tert-butyloxy, pentyloxy and hexyloxy. In a preferred embodiment, the “alkoxy” or “alkyloxy” is a methoxy.

The term “halogen” corresponds to a fluorine, chlorine, bromine, or iodine atom, preferably a fluorine, chlorine or bromine.

The expression “substituted by at least” means that the radical is substituted by one or several groups of the list. The expression “optionally substituted” means, without any otherwise precision, optionally substituted by a hydroxy, a halogen, and a (C₁-C₆)alkyl. A (C₁-C₆)alkyl optionally substituted by at least one halogen is preferably optionally substituted by at least one fluorine. For instance, a methyl optionally substituted by at least one fluorine includes CH₃, CH₂F, CHF₂, and CF₃.

The “stereoisomers” are isomeric compounds that have the same molecular formula and sequence of bonded atoms, but differ in the 3D-dimensional orientations of their atoms in space. The stereoisomers include enantiomers, diastereoisomers, Cis-trans and E-Z isomers, conformers, and anomers. In a preferred embodiment of the invention, the stereoisomers include diastereoisomers and enantiomers. The enantiomers compounds may be prepared from the racemate compound using any purification method known by a skilled person, such as LC/MS and chiral HPLC analysis methods and chiral SFC purification methods.

The “pharmaceutically acceptable salts” include inorganic as well as organic salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic, tartaric, and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromhydrate, methanesulfonate, and tartrate, preferably tartrate. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine. In a preferred embodiment, the salt is a tartrate salt.

As used herein, the terms “treatment”, “treat” or “treating” refer to any act intended to ameliorate the health status of patients such as therapy, prevention, prophylaxis and retardation of a disease, in particular a neuropsychiatric disorder. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to a mammal, more preferably to a human, including adult, child, adolescent, newborn and human at the prenatal stage. However, the term “subject” can also refer to non-human animals, in particular mammals such as dogs, cats, horses, cows, pigs, sheep and non-human primates, among others.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refers to a component of a pharmaceutical composition having a therapeutic effect.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “effective amount” refers to a quantity of an active ingredient or of a pharmaceutical composition which prevents, removes or reduces the deleterious effects of the disease, particularly a neuropsychiatric disorder. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

Compounds

The present invention provides trimeprazine derivatives of formula (I) as defined herein for use for treating a neuropsychiatric disorder such as an intellectual disability (ID), preferably fragile X syndrome (FXS), autism spectrum disorders (ASD), and attention deficit hyperactivity disorder (ADHD).

The present invention thus relates to a compound of formula (I):

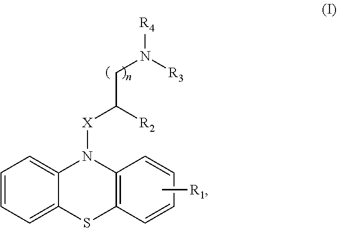

for use according to the invention, wherein:
X is —CO— or —CH₂—
n is 0 or 1;
R₁ represents:
   a hydrogen,
   a —CO—R₅ with R₅ being a hydrogen or a (C₁-C₆)alkyl,
   a (C₁-C₆)alkyl optionally substituted by at least one halogen,
   a (C₁-C₆)alkyloxy optionally substituted by at least one halogen,
   a halogen, or
   a —SO₂—NR₆R_{6′} with R₆ and R_{6′} being independently a hydrogen or a (C₁-C₆)alkyl;
R₂ represents a hydrogen or a (C₁-C₆)alkyl; and
R₃ and R₄ represent independently a hydrogen or a (C₁-C₆)alkyl.

In a preferred embodiment, a compound for use of formula (I) is such that:
X is —CO— or —CH₂—
n is 0 or 1;
R₁ represents:
   a hydrogen,
   a —CO—R₅ with R₅ being (C₁-C₃)alkyl, preferably a methyl or an ethyl,
   a methyl substituted by at least one fluorine, preferably a —CF₃,
   a methoxy,
   a chlorine, or
   a —SO₂—NR₆R_{6′} with R₆ and R_{6′} being a methyl;
R₂ represents a hydrogen or a methyl; and
R₃ and R₄ represent independently a (C₁-C₆)alkyl, preferably a methyl or an ethyl.

In a particular embodiment, X is —CH₂—, and n, R₁, R₂, R₃, and R₄ are such as defined herein.

In a further particular embodiment, n is 1, and X, R₁, R₂, R₃ and R₄ are such as defined herein.

In a further particular embodiment, R₁ is H, and n, X, R₂, R₃ and R₄ are such as defined herein.

In a further particular embodiment, R₂ is a methyl, and n, X, R₁, R₃ and R₄ are such as defined herein.

In a further particular embodiment, R₃ and R₄ represent a methyl, and n, X, R₁, and R₂ are such as defined herein.

In a preferred embodiment, a compound of formula (I) for use according to the invention is selected in a group consisting of trimeprazine, acepromazine, aceprometazine, chlorpromazine, dacemazine, dimetotiazine, fluacizine, levomeprazine, profenamine, promazine, promethazine, propiomazine, triflupromazine, and a pharmaceutical acceptable salt thereof. In a more preferred embodiment, said compound of formula (I) for use according to the invention is trimeprazine or one of its pharmaceutical acceptable salt.

Trimeprazine (also known as alimemazine), also called “SM4” herein, is a phenothiazine derivative of the following formula:

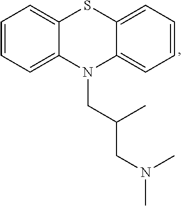 [SMILES: CC(CN(C)C)CN1c2ccccc2Sc2ccccc21]

currently used as an antipruritic. Trimeprazine is commonly provided as a tartrate salt. A more preferred compound of formula (I) for use according to the invention is thus trimeprazine tartrate.

Therapeutic Uses

As illustrated by examples, the inventors have demonstrated the therapeutic interest of compounds of formula (I) for treating a a neuropsychiatric disorder such as an intellectual disability (ID), preferably fragile X syndrome (FXS), autism spectrum disorders (ASD), and attention deficit hyperactivity disorder (ADHD).

Accordingly, the present invention relates to a compound of formula (I) and the pharmaceutically acceptable salts thereof as defined herein for use for treating a neuropsychiatric disorder chosen among an intellectual disability (ID), preferably fragile X syndrome (FXS), autism spectrum disorders (ASD), and attention deficit hyperactivity disorder (ADHD). Preferably, the compound of formula (I) is used a sole active ingredient.

As used herein, an “intellectual disability” is s a term used when a person has certain limitations in cognitive functioning and skills, including communication, social and self-care skills. These limitations can cause a child to develop and learn more slowly or differently than a typically developing child. One associated genetic disorder of “intellectual disability” is “Fragile X syndrome (FXS)”. Physical features of FXS may include a long and narrow face, large ears, flexible fingers, and large testicles. About a third of those affected have features of autism such as problems with social interactions and delayed speech. Hyperactivity is also common.

Attention-deficit/hyperactivity disorder (ADHD) is a disorder marked by an ongoing pattern of inattention and/or hyperactivity-impulsivity that interferes with functioning or development. Inattention can mean a person wanders off task, lacks persistence, has difficulty sustaining focus, and is disorganized; and these problems are not due to defiance or lack of comprehension. Hyperactivity can mean a person seems to move about constantly, including in situations in which it is not appropriate; or excessively fidgets, taps, or talks. In adults, it may be extreme restlessness or wearing others out with constant activity. Impulsivity can mean a person makes hasty actions that occur in the moment without first thinking about them and that may have a high potential for harm, or a desire for immediate rewards or inability to delay gratification. An impulsive person may be socially intrusive and excessively interrupt others or make important decisions without considering the long-term consequences. Inattention and hyperactivity/impulsivity are the key behaviors of ADHD. Some people with ADHD only have problems with one of the behaviors, while others have both inattention and hyperactivity-impulsivity. Most children have the combined type of ADHD. In preschool, the most common ADHD symptom is hyperactivity. It is normal to have some inattention, unfocused motor activity, and impulsivity, but for people with ADHD, these behaviors are more severe, occur more often, and interfere with or reduce the quality of social relationship.

In a preferred embodiment of the invention, said compound of formula (I) and pharmaceutically acceptable salts thereof as defined herein are for use for treating a neuropsychiatric disorder chosen among fragile X syndrome (FXS), autism spectrum disorders (ASD) and Attention deficit hyperactivity disorder (ADHD).

A further object of the invention is a pharmaceutical composition comprising a compound of formula (I), pharmaceutical acceptable salts thereof as defined herein, preferably trimeprazine, more preferably trimeprazine tartrate, and a pharmaceutically acceptable excipient, for use for treating a neuropsychiatric disorder chosen among an intellectual disability (ID), preferably fragile X syndrome (FXS), autism spectrum disorders (ASD), and attention deficit hyperactivity disorder (ADHD). In a preferred embodiment, said pharmaceutical composition comprises trimeprazine or one of its pharmaceutical acceptable salt as a sole active ingredient.

As used herein, the term “comprise(s)” or “comprising” (and other comparable terms, e.g., “containing,” and “including”) is “open-ended” and can be generally interpreted such that all of the specifically mentioned features and any optional, additional and unspecified features are included. According to specific embodiments, it can also be interpreted as the phrase “consisting essentially of” where the specified features and any optional, additional and unspecified features that do not materially affect the basic and novel characteristic(s) of the claimed invention are included or the phrase “consisting of” where only the specified features are included, unless otherwise stated.

Accordingly, another object of the invention is a pharmaceutical composition consisting essentially of or consisting of a compound of formula (I) and pharmaceutical acceptable salts thereof as defined herein, preferably trimeprazine, more preferably trimeprazine tartrate, for use for treating a neuropsychiatric disorder chosen among an intellectual disability (ID), preferably fragile X syndrome (FXS), autism spectrum disorders (ASD), and attention deficit hyperactivity disorder (ADHD).

A further object of the invention is also a method for treating a neuropsychiatric disorder selected in the group consisting of an intellectual disability (ID), preferably fragile X syndrome (FXS), autism spectrum disorders (ASD), and attention deficit hyperactivity disorder (ADHD), comprising administering an effective amount of a compound of formula (I) or one of its pharmaceutically acceptable salts as defined herein or a pharmaceutical composition as defined herein in a subject in need thereof.

A further object of the invention is a use of a compound of formula (I) or one of its pharmaceutically acceptable salts as defined herein for the manufacture of a pharmaceutical composition for treating a neuropsychiatric disorder selected in the group consisting of an intellectual disability (ID), preferably fragile X syndrome (FXS), autism spectrum disorders (ASD), and attention deficit hyperactivity disorder (ADHD).

According to the invention, the pharmaceutical composition for use comprises a pharmaceutically acceptable excipient. As used herein, the term “pharmaceutically acceptable excipient or carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

The compound for use according to the invention or the pharmaceutical composition for use according to the invention may be administered by any conventional route of administration. In particular, the compound or the pharmaceutical composition of the invention can be administered by a topical, enteral, oral, parenteral, intranasal, intravenous, intra-arterial, intramuscular, intratumoral, subcutaneous or intraocular administration and the like, preferably by oral or parenteral route, more preferably by intraperitoneal route. In particular, the compound for use according to the invention or the pharmaceutical composition for use according to the invention can be formulated for a topical, enteral, oral, parenteral, intranasal, intravenous, intra-arterial, intramuscular, intratumoral, subcutaneous or intraocular administration and the like.

Preferably, the compound for use according to the invention or the pharmaceutical composition for use according to the invention is administered by oral or parenteral route of administration.

When administered parenterally, the compound according to the invention or the pharmaceutical composition according to the invention is preferably administered by intraperitoneal route of administration. A preferred object of the invention is thus a compound for use according to the invention or a pharmaceutical composition for use according to the invention, which is administered by oral or parenteral route, preferably by intraperitoneal route.

The pharmaceutical composition comprising the molecule is formulated in accordance with standard pharmaceutical practice (Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

For oral administration, the composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatin, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants. For parenteral administration including intraperitoneal administration, the composition formulated conveniently comprises a sterile oily or aqueous preparation of the active ingredient which is preferably isotonic with the blood of the recipient. Every such formulation can also contain other pharmaceutically compatible and nontoxic auxiliary agents, such as stabilizers, antioxidants, binders, dyes, or emulsifiers.

The compound for use according to the invention or the pharmaceutical composition for use according to the invention may be administered as a single dose or in multiple doses. Preferably, the treatment is administered regularly, preferably between every day and every month, more preferably between every day and every two weeks, more preferably between every day and every week, even more preferably the treatment is administered every day.

The duration of treatment with the compound for use according to the invention or the pharmaceutical composition for use according to the invention is preferably comprised between 1 day and 20 weeks, more preferably between 1 day and 10 weeks, still more preferably between 1 day and 4 weeks, even more preferably between 1 day and 2 weeks. In a particular embodiment, the duration of the treatment is of about 1 week. Alternatively, the treatment may last as long as the disease persists.

The amount of the compound for use according to the invention or the pharmaceutical composition for use according to the invention to be administered has to be determined by standard procedure well known by those of ordinary skills in the art. Physiological data of the patient (e.g. age, size, and weight) and the routes of administration have to be taken into account to determine the appropriate dosage, so as a therapeutically effective amount will be administered to the patient.

In a preferred embodiment, the total compound dose for each administration of the compound for use according to the invention or of the pharmaceutical composition for use according to the invention is from 0.01 to 0.50 mg/kg BW, from 0.10 to 0.40 mg/kg BW, preferably from 0.20 to 0.30 mg/kg BW, 0.21 to 0.29 mg/kg BW, 0.22 to 0.28 mg/kg BW, 0.23 to 0.27 mg/kg BW, 0.24 to 0.26 mg/kg BW, and even more preferably at a dose of 0.25 mg/kg BW. As used herein “BW” means bodyweight. A preferred object of the invention is a pharmaceutical composition for use as defined herein, wherein said pharmaceutical composition is administered in a mammal at a dose of trimeprazine or one of its pharmaceutical acceptable salt ranging from 0.01 to 0.50 mg/kg BW, from 0.10 to 0.40 mg/kg BW, preferably from 0.20 to 0.30 mg/kg BW, even more preferably at a dose of 0.25 mg/kg BW. Such a specific dose of 0.25 mg/kg BW is up to 4-fold less than the dose currently used in the literature for treating other diseases than those of the present invention.

The form of the pharmaceutical compositions, the route of administration and the dose of administration of the compound according to the invention, or the pharmaceutical composition according to the invention can be adjusted by the man skilled in the art according to the type and severity of the disease, and to the patient, in particular its age, weight, sex, and general physical condition.

In preferred embodiment, the mammal is a human, preferably an infant, an adolescent, or an adult.

Further aspects and advantages of the present invention will be described in the following examples, which should be regarded as illustrative and not limiting.

### EXAMPLES

I. Material and Methods

Animals.

The experiments were performed following the ARRIVE (Animals in Research: Reporting In Vivo Experiments) guidelines. Fmr1-knockout (KO) and WT mice on a C57BL/6J congenic background were obtained from Prof. R. Willemsen. All animals were generated and housed in groups of 4 in standard laboratory conditions (22° C., 55±10% humidity, 12-h light/12-h dark diurnal cycles) with food and water provided ad libitum. Experimental testing was performed between 12:00 and 16:30 each day during the 12-h light period. Only male mice and rats were used. Animal care was conducted in accordance with the European Community Directive 2010/63/EU. The experiments were approved by the local ethics committee (Comité d′Ethique en Expérimentation Animale, by the French Ministry of Research.

Intraperitoneal Administration of SM2: Opipramol and SM4: Trimeprazine Drugs and Treatments.

Trimeprazine and opipramol were purchased from Prestwich Library. Both molecules were diluted in DMSO at the stock concentration of 100 mM. All compounds injected intraperitoneally were administered in a volume of 1.5 ml per kg body weight at the amount indicated in the legends of each figure.

Behavioural Tests.

Homing Behaviour Test: At PND 13, the litter was separated from the dam and kept for 30 min in a temperature-controlled holding cage. Then, each mouse pup was placed into a Plexiglas box whose floor was covered for ⅓ with bedding from the pup's home cage and for ⅔ with clean bedding. The pup was located at the side of the box covered by clean bedding, and its behaviour was videorecorded for 4 min for subsequent analysis. The following parameters were scored using the Observer 3.0 software (Noldus Information Technology): latency (s) to reach the home-cage bedding area; total time (s) spent by the pup in the nest bedding area.

Social Interaction Test: The 28-30-day-old mice were individually habituated to the experimental apparatus (a Plexiglas cage measuring 30×30×30 cm) for 5 min the day before testing. On the test day, the animals were isolated for 2 h before testing, to enhance their social motivation and thus facilitate the expression of social interaction during testing. The test consisted of placing 2 animals (same treatment and weight) into the test cage for 10 min. The behaviour of the animals was recorded using a video camera with zoom lens, DVD recorder and LCD monitor. Behaviour was assessed per single animal and analyzed by a trained observer who was unaware of genotype and treatment conditions using the Observer XT software (Noldus, The Netherlands).

The following parameters were scored:

a. Social Activities:
1. Social sniffing: sniffing any part of the body of the partner, including the anogenital area.
2. Following: moving in the direction of or pursuing the partner, who moves away.
3. Mutual circle: partners are mutually sniffing each other's anogenital region, while describing tight circles with their reciprocal following movements.
4. Pushing past: the focal animal passes between the wall of the cage and the body of the partner by pushing its own body through the narrow space available.
5. Crawling under/over: the focal animal crawls underneath or over the partner's body, crossing it transversely from one side to the other.
6. Social grooming: chewing and licking the fur of the partner.
7. Social rest: the focal animal is being groomed by the partner.
8. Pushing under: the focal animal pushes its own snout or the whole anterior part of its body under the partner's body, and rests for at least 3 s.
9. Social inactivity: the focal animal is lying flat or standing still (eyes closed or open) while maintaining close physical contact with the partner.

b. Nonsocial Activities:
1. Running: the focal animal performs a sudden, rapid, vigorous, and erratic darting, characterized by frequent and sharp changes in direction and without any obvious target.
2. Inactive: Self-explanatory.
3. Exploring: Self-explanatory.
4. Digging: the focal animal is digging in the sawdust, pushing and kicking it around, using the snout and/or both the forepaws and hindpaws.

The average frequency of total social activities, quantified as number of events during the 10 min testing session, was graphed.

Novel Object Recognition

On day 1 and 2, mice were habituated for 10 min to the maze in which the task was performed. On the third day, mice were put back in the maze for 10 min, two identical objects were presented and the time that the mice spent exploring each object was recorded. The mice were again placed in the maze 5 minutes later for 10 min, one of the familiar objects was replaced with a novel object and the total time spent exploring each of the two objects (novel and familiar) was computed. Object exploration was defined as the orientation of the nose to the object at a distance of less than 2 cm. A discrimination index was calculated as the difference between the times spent exploring either the novel or familiar object divided by the total time exploring the two objects. A higher discrimination index is considered to reflect greater memory retention for the familiar object. Pharmacological treatment with SM2 and SM4 was performed 30 minutes before training.

Statistical Analysis

Results are expressed as mean±standard error of the mean (SEM). All statistical analyses were based on biological replicates. Appropriate statistical tests used for each experiment are described in the corresponding figure legends. All statistical analyses were carried out using the GraphPad PrismVersion 6.0e.

II. Results

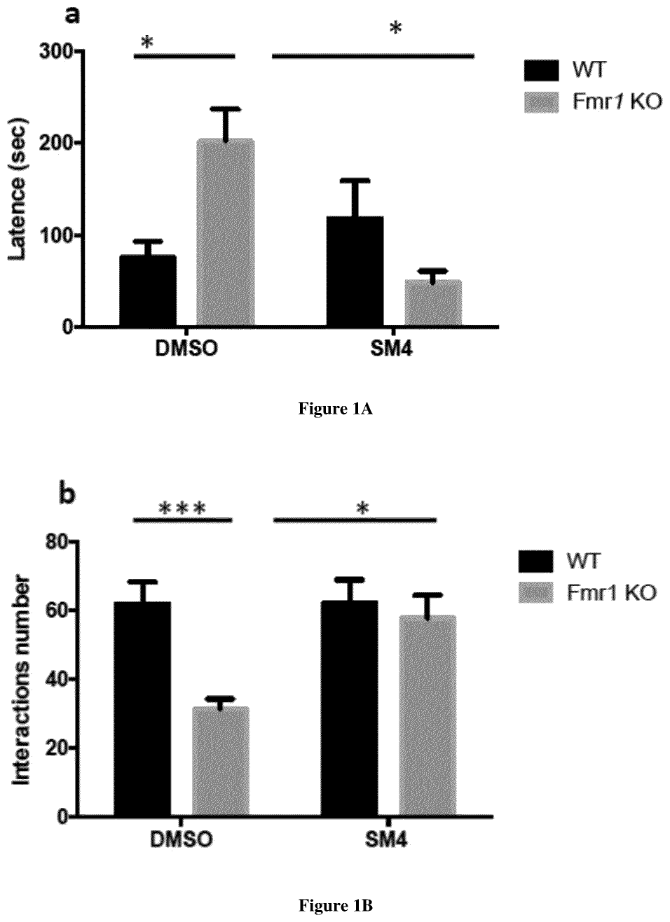

FIG. 1 displays the impact of SM4 on the social behaviour of infant and adolescent Fmr-KO mice (the model of Fragile X Syndrome). Two behaviours were studied: social discrimination, measured by the “homing test” (FIG. 1A) and social interaction, measured by the social interaction test (FIG. 1B°.

FIG. 1A: The latency time (the time that the mouse pups need to find the mother's litter) is longer in Fmr1-KO mice compared with wild-type mice (WT). This latency time is normalized in Fmr1-KO mice after treatment with SM4 and is not significantly different from the one of treated WT mice.

FIG. 1B: The number of social interactions is higher in WT mice compared with Fmr1-KO mice. This number of interactions is normalized after an acute treatment with the SM4 drug. This drug does not modify the social interaction behaviour of treated WT mice.

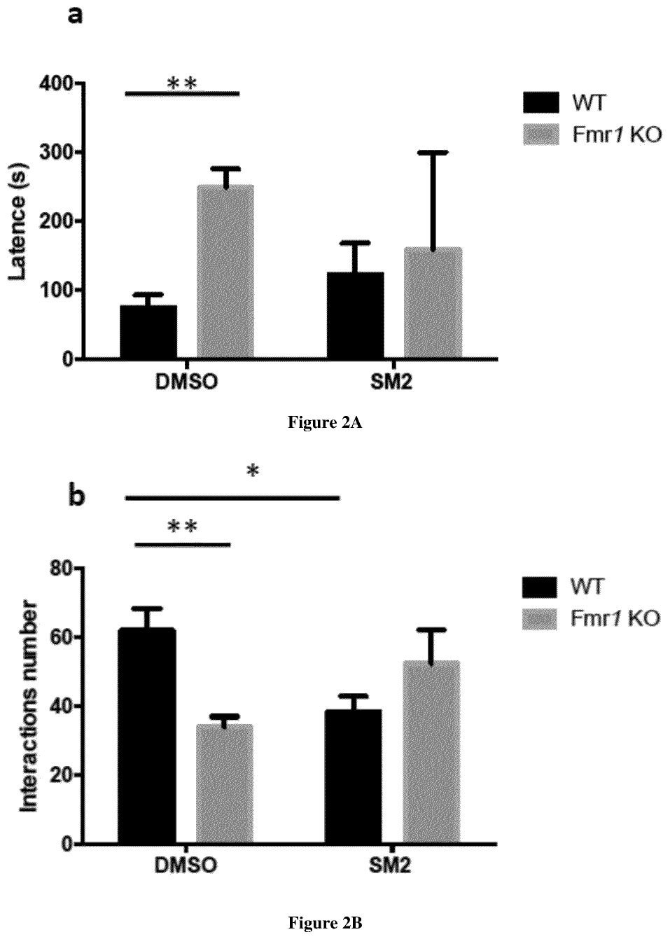

FIG. 2 displays the impact of SM2 on the social behaviour of infant and adolescent Fmr1-KO mice. Two behaviours are studied: social discrimination measured by the “homing test” (FIG. 2A) and social interaction, measured by the social interaction test (FIG. 2B). In both cases the treated mice (WT and Fmr1-KO) show the same behaviour as the untreated Fmr-KO mice. This means that, on one hand, SM2 does not have a beneficial impact on Fmr1-KO mice and, on the other hand, that SM2 has a negative impact on the behaviour of the WT mice.

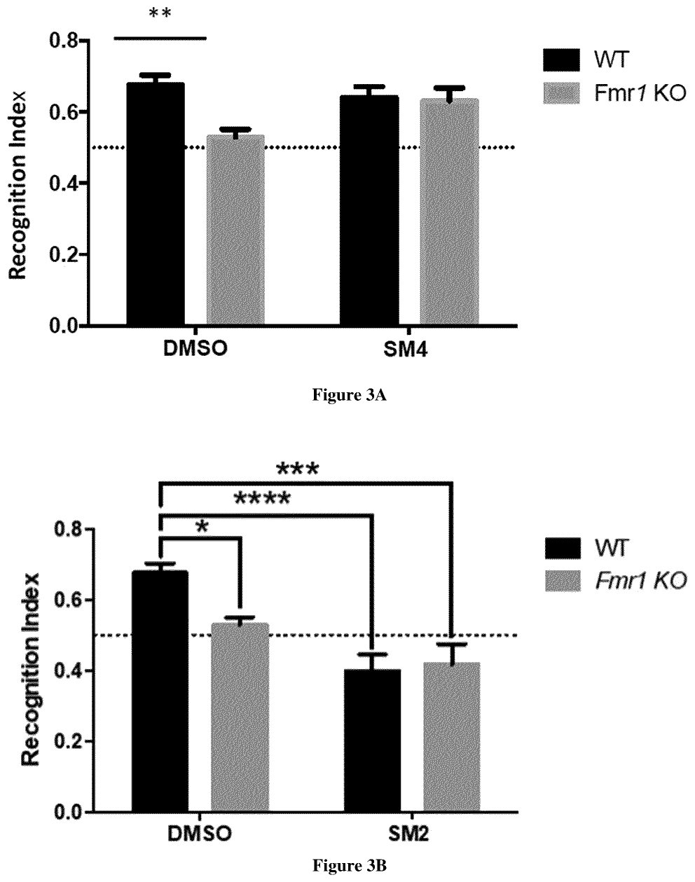

FIG. 3 displays the impact of SM4 (FIG. 3A) and SM2 (FIG. 3B) on the working memory of Fmr1-KO mice by using the novel object recognition test. In FIG. 3A, the untreated (receiving only DMSO) Fmr-KO mice showed a discrimination index significantly lower than untreated WT mice. Discrimination index is the same in treated WT and Fmr-KO mice. Interestingly, SM4 has no impact on the behaviour of treated WT mice. In FIG. 3B, Fmr1-KO mice were not normalized after treatment with the SM2 drug. Furthermore, SM2 worsened the behaviour of WT mice.

In conclusion, such results unexpectedly show an improved impact of trimeprazine on social behaviour and cognition, demonstrating thereby a higher efficacy of trimeprazine and derivatives thereof for treating a neuropsychiatric disorder, such as an intellectual disability (ID), preferably fragile X syndrome (FXS), autism spectrum disorders (ASD), and attention deficit hyperactivity disorder (ADHD).

1-15. (canceled)

16. A method of treating a neuropsychiatric disorder comprising administering a compound of formula (I): 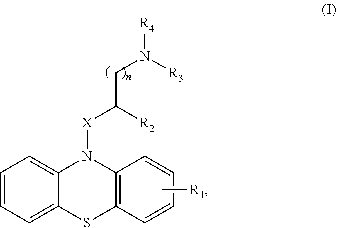
wherein:
X is —CO— or —CH₂—;
n is 0 or 1;
R₁ represents:
a hydrogen,
a —CO—R₅ with R₅ being a hydrogen or a (C₁-C₆)alkyl,
a (C₁-C₆)alkyl optionally substituted by at least one halogen,
a (C₁-C₆)alkyloxy optionally substituted by at least one halogen,
a halogen, or
a —SO₂—NR₆R_{6′} with R₆ and R_{6′} being independently a hydrogen or a (C₁-C₆)alkyl;
R₂ represents a hydrogen or a (C₁-C₆)alkyl; and
R₃ and R₄ represent independently a hydrogen or a (C₁-C₆)alkyl;
a stereoisomer thereof, a pharmaceutically acceptable salt thereof, or a pharmaceutical composition comprising said compound, stereoisomer, or pharmaceutically acceptable salt to a subject in need of treatment.

17. The method according to claim 16, wherein said neuropsychiatric disorder is an intellectual disability (ID), fragile X syndrome (FXS), autism spectrum disorder (ASD), or attention deficit hyperactivity disorder (ADHD).

18. The method according to claim 17, wherein said neuropsychiatric disorder is FXS or ASD.

19. The method according to claim 16, wherein:
X is —CO— or —CH₂—;
n is 0 or 1;
R₁ represents:
a hydrogen,
a —CO—R₅ with R₅ being (C₁-C₃)alkyl,
a methyl substituted by at least one fluorine,
a methoxy,
a chlorine, or
a —SO₂—NR₆R_{6′} with R₆ and R_{6′} being a methyl;
R₂ represents a hydrogen or a methyl; and
R₃ and R₄ represent independently a (C₁-C₆)alkyl.

20. The method according to claim 19, wherein R₁ is —CO—R₅ with R₅ being a methyl or an ethyl or —CF₃.

21. The method according to claim 16, wherein X is —CH₂—.

22. The method according to claim 16, wherein n is 1.

23. The method according to claim 16, wherein R₁ is H.

24. The method according to claim 16, wherein R₂ is a methyl.

25. The method according to claim 16, wherein R₃ and R₄ represent a methyl or an ethyl.

26. The method according to claim 25, wherein R₃ and R₄ represent a methyl.

27. The method according to claim 16, wherein said compound is selected from the group consisting of trimeprazine, acepromazine, aceprometazine, chlorpromazine, dacemazine, dimetotiazine, fluacizine, levomeprazine, profenamine, promazine, promethazine, propiomazine, triflupromazine, and a pharmaceutical acceptable salt thereof.

28. The method according to claim 27, wherein said compound is trimeprazine or a pharmaceutically acceptable salt thereof.

29. The method according to claim 28, wherein the pharmaceutically acceptable salt is trimeprazine tartrate.

30. The method according to claim 16, wherein the method comprises administering a pharmaceutical composition comprising trimeprazine or one of its pharmaceutical acceptable salt as a sole active ingredient.

31. The method according to claim 16, wherein said pharmaceutical composition is administered to a mammal at a dose of trimeprazine or one of its pharmaceutical acceptable salt ranging from 0.01 to 0.50 mg/kg BW.

32. The method according to claim 31, wherein said mammal is a human adult, human infant, or human adolescent.

33. The method according to claim 16, wherein said pharmaceutical composition is administered by oral or parenteral route.

34. The method according to claim 33, wherein said pharmaceutical composition is administered intraperitoneally.
